(19) United States
(12) Patent Application Publication
(10) Pub. No.: US 2026/0286330 A1
(43) Pub. Date: Sep. 24, 2026

(54) COMPOSITIONS COMPRISING MODIFIED HUMAN GALACTOCEREBROSIDASE

(71) Applicant: M6P THERAPEUTICS, INC., St. Louis, MO (US)

(72) Inventors: Lin LIU, Ballwin, MO (US); Andrew Charles HEDMAN, St. Louis, MO (US)

(21) Appl. No.: 19/474,313

(22) PCT Filed: Mar. 28, 2024
(86) PCT No.: PCT/US24/22007

§ 371 Date: Oct. 10, 2025

Related U.S. Application Data
(60) Provisional application No. 63/458,257, filed on Apr. 10, 2023.

(51) Int. Cl. C12N 9/24

(52) U.S. Cl. CPC C12N 9/2402; C12Y 302/01046

(57) ABSTRACT

Provided are modified forms of human Galactocerebrosidase (GALC) enzyme. Included are descriptions of the methods for synthesis of DNA constructs for GALC for the expression of the GALC enzyme with modified amino acid sequence. Additionally, descriptions of GALC enzyme assays are provided that demonstrate higher specific GALC activity for proteins with the modified GALC amino acid sequence.

### CROSS REFERENCE TO RELATED APPLICATIONS

This application claims the benefit of priority to U.S. Patent Application Ser. No. 63/458,257, filed Apr. 10, 2023, the entire contents of which are incorporated herein by reference.

### BACKGROUND

Krabbe disease is a neurological disorder caused by loss of Galactocerebrosidase (GALC) enzyme activity [1]. Lack of functional GALC results in the accumulation of substrates, such as Galactocerebroside and Galactosylsphingosine, that will result in impaired neuron myelination, leading to loss of motor and neurological functions, eventually causing death (reviewed in [2]). Described here for this invention are the amino acid sequences for a modified human GALC enzyme with enhanced enzyme activity.

### SUMMARY

The present disclosure is directed to a composition comprising a modified whole or truncated wild type GALC comprising amino acid substitutions that result in enhanced enzyme activity. In one embodiment, the amino acid substitutions comprise one or more of the following: T145 to I145, T177 to R177, S200 to P200, 1208 to E208, S232 to P232, K261 to W261, Y300 to N300, and C322 to S322. In an embodiment, the composition comprises the amino acid sequence of SEQ ID NO. 2. In an embodiment, the evidences increased GALC enzyme activity compared to wild-type GALC. In an embodiment, the increased GALC enzyme activity comprises at least 2-fold higher GALC enzyme activity compared to wild-type GALC.

The present disclosure is also directed to a composition comprising a modified whole or truncated wild type human GALC comprising the amino acid sequence of SEQ ID NO. 1, comprising one or more of the following amino acid substitutions: T145 to 1145, T177 to R177, S200 to P200, 1208 to E208, S232 to P232, K261 to W261, Y300 to N300, and C322 to S322. In an embodiment, the composition comprises a human GALC enzyme having amino acid substitutions of S200 to P200 and S232 to P232. In an embodiment, the composition comprises increased GALC enzyme activity. In an embodiment, the increased GALC enzyme activity comprises around 2-fold to 9-fold higher GALC enzyme activity compared to wild-type GALC. In an embodiment, the composition comprises a human GALC enzyme having an amino acid substitution Y300 to N300. In an embodiment, the composition comprises an additional glycosylation site compared to wild-type GALC. In an embodiment, the composition comprises a human GALC enzyme having one or more amino acid substitutions of T177 to R177, 1208 to E208, K261 to W261, and Y300 to N300. In an embodiment, the composition comprises stabilized enzyme structure compared to wild-type GALC. In an embodiment, the composition comprises one or more amino acid substitutions of S200 to P200, S232 to P232, and S232 to P232. In an embodiment, the composition comprises a more rigid active site loop compared to wild-type GALC.

In any of the foregoing embodiments, the wild-type GALC comprises the amino acid sequence of SEQ ID NO. 1.

### BRIEF DESCRIPTION OF THE DRAWINGS

FIG. 1A depicts in vitro GALC enzyme activity of conditioned media containing GALC-M2N6 or GALC-WT expressed by transfection of Expi293T cells.

FIG. 1B depicts via Western Blot the amount of GALC-M2N6 and GALC-WT in transfected Expi293T cells.

FIG. 2A depicts Coomassie-stained SDS-PAGE gel loaded with purified GALC-WT and GALC-M2N6 protein.

FIG. 2B depicts Coomassie-stained SDS-PAGE gel loaded with an amount equal to the activity found in wild-type GALC.

FIG. 2C depicts GALC enzyme activity of purified recombinant GALC-M2N6 or GALC-WT.

FIG. 3A depicts GALC enzyme activity of GALC-WT, GALC-M2N6, and GALC with individual point mutations.

FIG. 3B depicts via Western Blot the amount of GALC-WT, GALC-M2N6, and GALC with individual point mutations.

FIG. 4 depicts the GALC enzyme activity of the GALC-WT, GALC-M2N6, S200P, S232P, and combined S200P/S232P construct.

### DETAILED DESCRIPTION

The invention comprises specific modified forms of the human GALC enzyme that demonstrate enhanced enzyme activity and/or other properties, including stability and/or glycosylation. In one aspect, eight amino acid residues are substituted in a wild type human GALC sequence [NCBI Reference Sequence: NP_000144.2]. The original human amino acid sequence for human GALC and the modified human M2N6 GALC sequences are shown respectively as SEQ ID NO. 1 (wild-type human GALC) and SEQ ID NO. 2 (modified human GALC having eight AA substitutions, which is sometimes referred to herein is as M2N6 GALC).

SEQ ID No. 1:
Human wild-type GALC> 685 amino acids
MAEWLLSASWQRRAKAMTAAAGSAGRAAVPLLLCALLAPGGAYVLDDSDG
LGREFDGIGAVSGGGATSRLLVNYPEPYRSQILDYLFKPNFGASLHILKV
EIGGDGQTTDGTEPSHMHYALDENYFRGYEWWLMKEAKKRNPNITLIGLP
WSFPGWLGKGFDWPYVNLQLTAYYVVTWIVGAKRYHDLDIDYIGIWNERS
YNANYIKILRKMLNYQGLQRVKIIASDNLWESISASMLLDAELFKVVDVI
GAHYPGTHSAKDAKLTGKKLWSSEDFSTLNSDMGAGCWGRILNQNYINGY
MTSTIAWNLVASYYEQLPYGRCGLMTAQEPWSGHYVVESPVWVSAHTTQF
TQPGWYYLKTVGHLEKGGSYVALTDGLGNLTIIIETMSHKHSKCIRPFLP
YFNVSQQFATFVLKGSFSEIPELQVWYTKLGKTSERFLFKQLDSLWLLDS
DGSFTLSLHEDELFTLTTLTTGRKGSYPLPPKSQPFPSTYKDDFNVDYPF
FSEAPNFADQTGVFEYFTNIEDPGEHHFTLRQVLNQRPITWAADASNTIS
IIGDYNWTNLTIKCDVYIETPDTGGVFIAGRVNKGGILIRSARGIFFWIF
ANGSYRVTGDLAGWIIYALGRVEVTAKKWYTLTLTIKGHFTSGMLNDKSL
WTDIPVNFPKNGWAAIGTHSFEFAQFDNFLVEATR.
SEQ ID NO. 2:
Modified Human M2N6 GALC> 685 amino acids
MAEWLLSASWQRRAKAMTAAAGSAGRAAVPLLLCALLAPGGAYVLDDSDG
LGREFDGIGAVSGGGATSRLLVNYPEPYRSQILDYLFKPNFGASLHILKV
EIGGDGQTTDGTEPSHMHYALDENYFRGYEWWLMKEAKKRNPNIILIGLP
WSFPGWLGKGFDWPYVNLQLTAYYVVRWIVGAKRYHDLDIDYIGIWNERP
YNANYIKELRKMLNYQGLQRVKIIASDNLWEPISASMLLDAELFKVVDVI
GAHYPGTHSAWDAKLTGKKLWSSEDFSTLNSDMGAGCWGRILNQNYINGN
MTSTIAWNLVASYYEQLPYGRSGLMTAQEPWSGHYVVESPVWVSAHTTQF
TQPGWYYLKTVGHLEKGGSYVALTDGLGNLTIIIETMSHKHSKCIRPFLP
YFNVSQQFATFVLKGSFSEIPELQVWYTKLGKTSERFLFKQLDSLWLLDS
DGSFTLSLHEDELFTLTTLTTGRKGSYPLPPKSQPFPSTYKDDFNVDYPF
FSEAPNFADQTGVFEYFTNIEDPGEHHFTLRQVLNQRPITWAADASNTIS
IIGDYNWTNLTIKCDVYIETPDTGGVFIAGRVNKGGILIRSARGIFFWIF
ANGSYRVTGDLAGWIIYALGRVEVTAKKWYTLTLTIKGHFTSGMLNDKSL
WTDIPVNFPKNGWAAIGTHSFEFAQFDNFLVEATR

Modified M2N6 GALC contains 8 amino acid substitutions in the human GALC sequence. These modifications include (in one letter amino acid abbreviations): (1) T145 to I145, (2) T177 to R177, (3) S200 to P200, (4) I208 to E208, (5) S232 to P232, (6) K261 to W261, (7) Y300 to N300, (8) C322 to S322. These residues are underlined in both sequences. To further evaluate the effectiveness of each amino acid involved, single point mutation constructs were generated. Data indicate the S200P and S232P mutations are key amino acids to increase the GALC enzyme activity. The Y300N mutation generates an additional glycosylation site in the enzyme.

### EXAMPLES

The disclosure is described with reference to the following Examples. These Examples are provided for the purpose of illustration only and the disclosure should in no way be construed as being limited to these Examples, but rather should be construed to encompass any and all variations which become evident as a result of the disclosure provided herein.

Without further description, it is believed that one of ordinary skill in the art can, using the preceding description and the following illustrative examples, make and utilize the compounds of the present disclosure and practice the claimed methods. The following working examples are not to be construed as limiting in any way the present disclosure.

### Example 1

### Methods

Molecular cloning: The cDNA for human GALC was obtained from Origene (product number RC211578, cDNA related to NM_000153). Also, codon-optimized GALC was obtained from Integrated DNA Technologies (IDT) by using gene-synthesis and provided codon optimization software on the IDT website (https://www.idtdna.com/CodonOpt). In parallel, human GALC regions were synthesized in custom DNA gBlocks (https://www.idtdna.com/pages/products/genes-and-gene-fragments/double-stranded-dna-fragments/gblocks-gene-fragments), that allowed for assembly of the modified GALC cDNA containing the multiple mutations to alter the codons at the specified positions. Nucleic acid sequences for the generation of GALC N6 and M2 mutations are shown in SEQ ID NO. 3 and SEQ ID NO. 4. Additionally, site-directed mutagenesis was performed using primer overlap extension followed by DpnI treatment to modify individual residues. DNA fragments containing the GALC point mutations and expression plasmids were placed into pcDNA3.1 based vectors, containing a C-terminal HPC4 tag (protein C derived amino acid sequence SGRGEDQVDPRLIDGK; [3,4] for protein expression using standard molecular biology techniques (restriction digest, ligation, bacterial transformation).

SEQ ID NO. 3:
>GALC N6 Nhel-HindIII hGALC(T145I, T177R, S200P,
I208E, S232P, K261W)-fragment (Nhel&HindIII)
acGCTAGCCACCatggctgagtggctactctcggcttcctggcaacgccg
agcgaaagctatgactgctgcAgcTggttcggcAggAAgAgcTgcTgtgc
ccttgctgctgtgtgcgctgctggcTccTggTggAgcAtacgtgctcgac
gactccgacgggctgggccgggagttcgacggcatcggcgcggtcagcgg
cggcggggcaacctcccgacttctagtaaattacccagagccctatcgtt
ctcagatattggattatctctttaagccgaattttggtgcctctttgcat
attttaaaagtggaaataggtggtgatgggcagacaacagacggcactga
gccctcccacatgcattatgcactagatgagaattatttccgaggatacg
agtggtggttgatgaaagaagctaagaagaggaatcccaatattaTCctc
attgggttgccatggtcattccctggatggctgggaaaaggtttcgactg
gccttatgtcaatcttcagctgactgcctattatgtcgtgaGAtggattg
tgggcgccaagcgttaccatgatttggacattgattatattggaatttgg
aatgagaggCcatataatgccaattatattaagGAGttaagaaaaatgct
gaattatcaaggtctccagcgagtgaaaatcatagcaagtgataatctct
gggagCccatctctgcatccatgctccttgatgccgaactcttcaaggtg
gttgatgttataggggctcattatcctggaacccattcagcaTGGgatgc
aaagttgactgggaagAAGCTTtg
SEQ ID NO. 4:
>GALC M2 HindIII AflII hGALC(Y300N,C322S)-fragment
(HindIII&AflII)
aagaagctttggtcttctgaagactttagcactttaaatagtgacatggg
tgcaggctgctggggtcgcattttaaatcagaattatatcaatggcAata
tgacttccacaatcgcatggaatttagtggctagttactatgaacagttg
ccttatgggagaAgcgggttgatgacggcccaggagccatggagtgggca
ctacgtggtagaatctcctgtctgggtatcagctcataccactcagttta
ctcaacctggctggtattacctgaagacagttggccatttagagaaagga
ggaagctacgtagctctgactgatggcttagggaacctcaccatcatcat
tgaaaccatgagtcataaacattctaagtgcatacggccatttcttcctt
atttcaatgtgtcacaacaatttgccacctttgttcttaaggg

Cell culture: HEK293T (ATCC CRL-3216) were cultured under Dulbecco's Modified Eagle's medium (DMEM) with 10% heat inactivated FBS (ATCC 30-2020) and 2 mM L-glutamine (ThermoFisher 25030081). Expi293F (Gibco A14527) cells were grown in the Expi293 expression media (Gibco A1435101).

Protein expression: Expi293F cells (Gibco A14527) grown in Expi293F expression media in suspension culture were transfected using Expifectamine reagent (Gibco A14525) following manufacturer's protocol. Cells and conditioned media were harvested 72 h after transfection. Conditioned media was collected and centrifuged to remove debris. Cells were rinsed in PBS, lysed in M-PER buffer (Thermo 78501) containing 1% protease and phosphatase inhibitor cocktail (MidSci IB01070), sonicated, and centrifuged to remove debris. Conditioned media and cell lysate were then used for GALC activity assays, protein purification and SDS-PAGE analysis.

Protein purification: HPC4-tagged GALC proteins were purified from conditioned media using Anti-Protein C Affinity Matrix (Roche, 11815024001), columns were washed, and proteins eluted using Tris-EDTA buffer, following the manufacturer's protocol.

GALC activity assay: GALC activity was measured following an established protocol ([5]. Briefly, assays were performed in 0.1M-0.2M Citrate-Phosphate buffer, pH4.0, 0.25% TX-100. GALC substrate was 6-Hexadecanoylamino-4-methylumbelliferyl β-D-galactopyranoside (HMGAL), (Carbosynth EH05989) dissolved in DMSO at 10 mM concentration and diluted to final 1 mM in assay buffer for activity assay. For the assays, sodium taurocholate (Biosynth S-6500, final concentration 6 mg/ml) and oleic acid (Millipore-Sigma O1008 final concentration 0.2%) were added to the substrate solution. Substrate solution was sonicated at 50% power for 6 seconds. 45 μl of substrate solution was aliquoted into a 96 well plate, and 5 μl of sample (cell lysate, conditioned media, purified protein, etc.) were added in duplicate (2 wells per sample). To compare activity, serial dilutions of 6-Hexadecanoylamino-4-methylumbelliferone (6-HM) (Carbosynth EH10520) standards were incubated in the same plate during the assay. The plate was then sealed and incubated at 37° C. for 1 hr with shaking/agitation. Reactions were stopped by addition of 150 μl of stop buffer (0.4 M glycine-NaOH, pH 10.8.) GALC activity was then measured using substrate fluorescence at Ex 360, Em 460 using a Molecular Devices Spectramax ID3 plate reader and Softmax Pro 7.1 software. Background fluorescence was subtracted based on substrate alone or mock transfection conditions. Analysis was performed in Excel and graphs generated using GraphPad Prism software. Protein concentrations for samples were measured using BCA protein assay (ThermoFisher 23227) following the manufacturer's protocol. Absorbance at 260 was then measured using a Molecular Devices Spectramax Id3 plate reader and Softmax Pro 7.1 software. Concentrations were compared to BSA standards.

SDS-PAGE and Western Blotting—Samples were boiled in protein loading buffer and examined on NuPage 4-12% Gradient Bis-Tris polyacrylamide gels. Gels were then stained using Coomassie Stain or transferred to nitrocellulose membrane. Blots were blocked in 5% Milk in PBST. Blots were probed with anti-HPC4 tag antibodies (Genscript, A01774) and HRP-tagged sheep anti-mouse secondary antibodies (ECL, NA931V). Blots were incubated with ECL substrate. Images of Coomassie stained gels or Western blots were captured using an Azure 400 Imaging System.

### Results

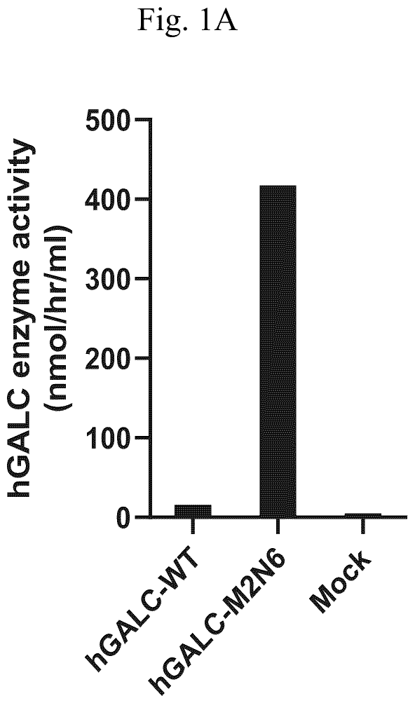

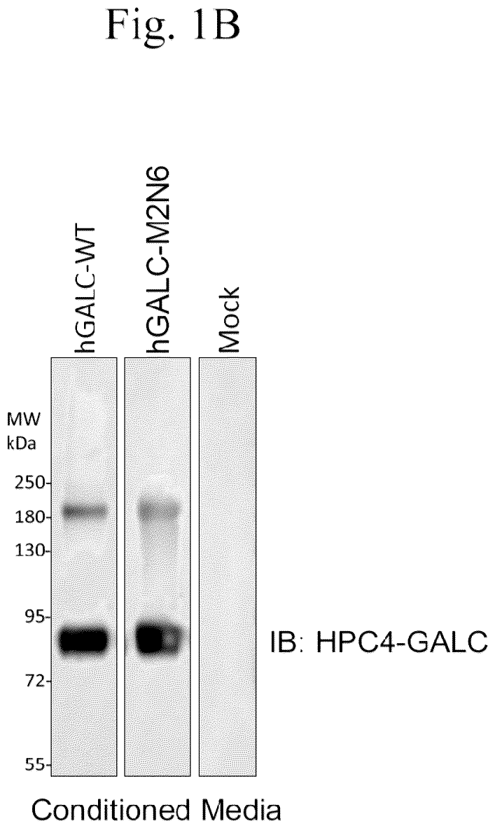

Conditioned media from GALC-WT or GALC-M2N6 transfected HeK293F cells was used for a GALC activity assay. The GALC-WT and GALC-M2N6 proteins were tagged with HPC4. The media of transfected cells was incubated with fluorescently labeled GALC substrate for 1 hour. Reactions were stopped and fluorescence was read on a plate reader. Conditioned media containing GALC-M2N6 has higher GALC enzyme activity (FIG. 1A-plotting GALC enzyme activity relative to the amount of protein incubated in the reaction). By Western blot, with anti-HPC4 antibody, similar amounts of HPC tagged GALC (WT or M2N6) are detectable in the conditioned media, with no HPC4 GALC expressed in media from Mock transfected cells (FIG. 1B).

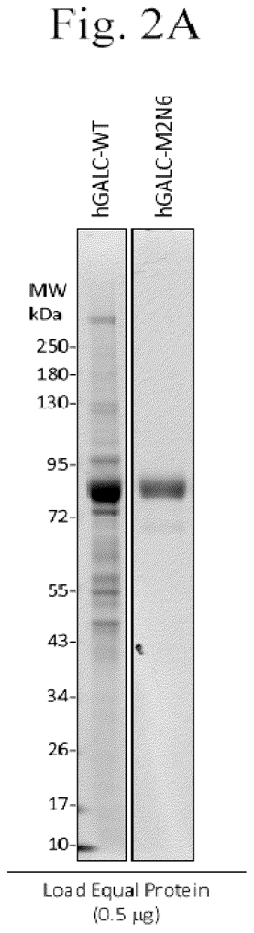

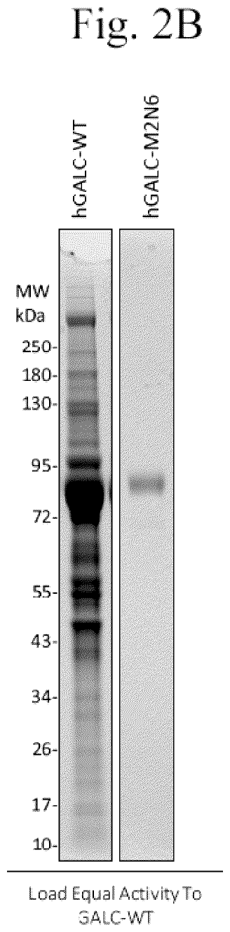

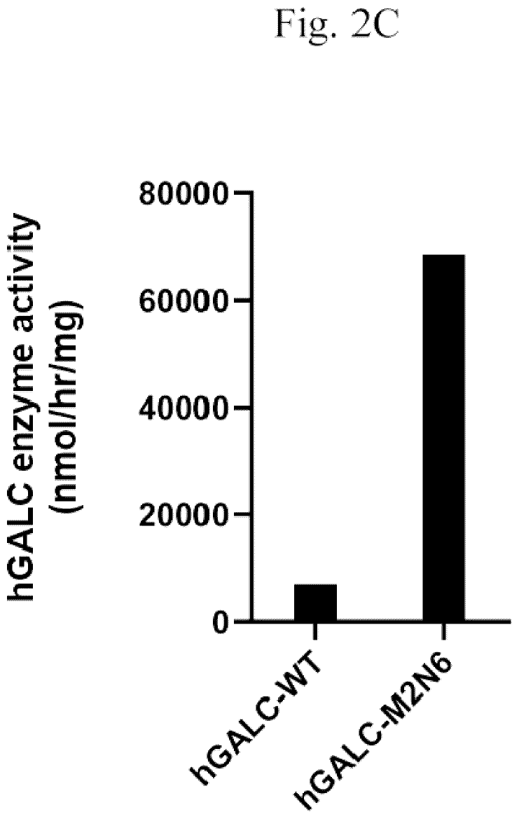

In addition, GALC enzyme activity was assessed for purified recombinant GALC (WT and M2N6) by HPC4 affinity purification. We observe that GALC M2N6 has higher specific enzyme activity than GALC WT (FIG. 2C). The purified proteins for wild-type and modified GALC were analyzed by Coomassie stained SDS-PAGE gels. Gels were loaded based on purified protein amount (FIG. 2A, 0.5 mg protein loaded) or an amount equal to the activity found in GALC-WT GALC (FIG. 2B). This result shows better purity and higher specific activity for the modified GALC.

To demonstrate which amino acids are the key ones in the construct of hGALC-M2N6 to maintain the high GALC activity, structure analysis was performed in the mutations involved. 4 of them (T177R, I208E, K261W and Y300N) were predicted to stabilize the enzyme structure and 3 of them (S200P, S232P and C322S) were indicated to rigidify the region of GALC that mediates catalytic activity (Table 1) based on structure analysis.

TABLE 1
List of GALC mutations and its potential role
in the regulation of enzyme function.

| Plasmid | Mutation | Prediction |
|---|---|---|
| G0306 | \ | |
| G0341 | M2N6 (T145I, T177R, | |
| | S200P, I208E, C322S, | |
| | S232p, K261W and | |
| | Y300N) | |
| G0343 | T177R | Stabilizing (salt bridge) |
| G0344 | S200P | Activating (rigidifying active site loop) |
| G0345 | I208E | Stabilizing (salt bridge) |
| G0346 | C322S | Activating (near active site) |
| G0348 | T145I | Not sure |
| G0349 | S232P | Activating (rigidifying active site loop) |
| G0350 | K261W | Stabilizing (with Y300N) |
| G0351 | Y300N | Stabilizing (with K261W) |

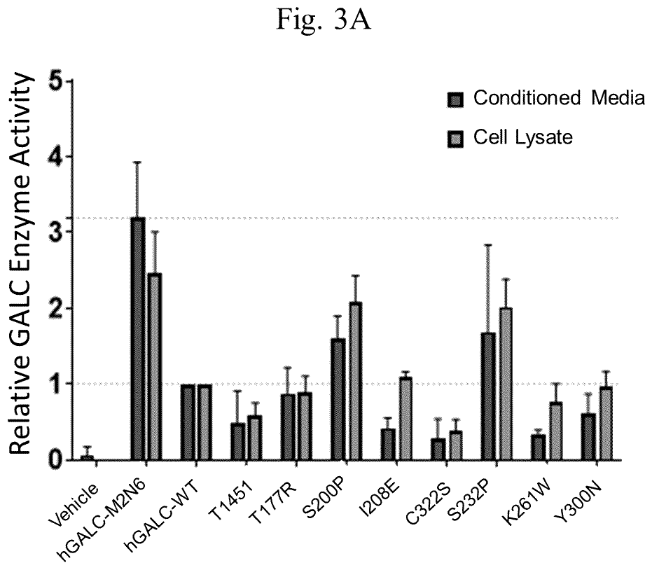

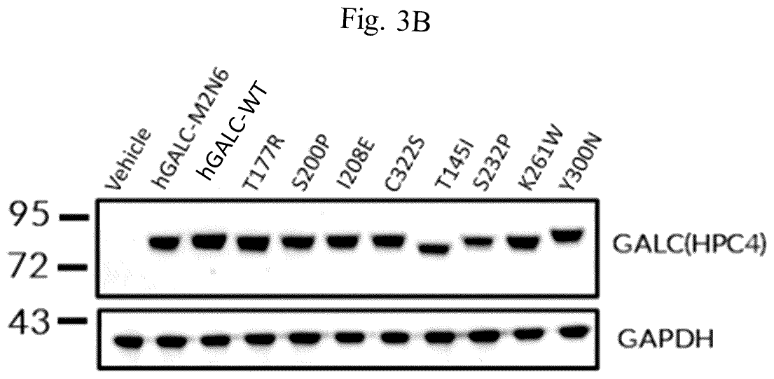

Eight individual point mutation constructs were generated by single-point mutation. The only single point mutations observed to increase GALC enzyme activity in conditioned medium and cell were S200P and S232P (FIG. 3A). Western blotting was also examined to check protein expression in cell (FIG. 3B). As data shown in FIG. 3B, T145I mutation could remove one of the N-glycosylation sites from GALC enzyme and run slightly fast in the SDS-PAGE gel. But Y300N can add one more glycosylation site as the enzyme molecular weight went up (FIG. 3B).

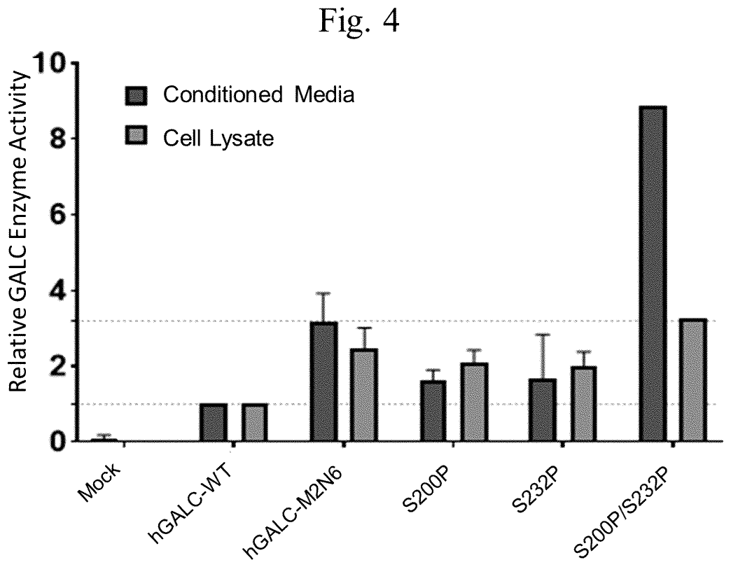

The combination of S200P/S232P construct shows the greatest increase of GALC enzyme activity in HEK293 cells (FIG. 4). In conclusion, we have identified a few mutations in human GALC enzyme which could increase human GALC enzyme activity by either increase enzyme stability at 37 C or higher pH (pH7.2). Thermo-stability and pH-stability studies to further characterize the GALC variants are ongoing.

### REFERENCES

Suzuki, K. and Y. Suzuki, Globoid cell leucodystrophy (Krabbe's disease): deficiency of galactocerebroside beta-galactosidase. Proc Natl Acad Sci USA, 1970. 66(2): p. 302-9.
Won, J. S., A. K. Singh, and I. Singh, Biochemical, cell biological, pathological, and therapeutic aspects of Krabbe's disease. J Neurosci Res, 2016. 94(11): p. 990-1006.
Lichty, J. J., et al., Comparison of affinity tags for protein purification. Protein Expr Purif, 2005. 41(1): p. 98-105.
Stearns, D. J., et al., The interaction of a Ca2+-dependent monoclonal antibody with the protein C activation peptide region. Evidence for obligatory Ca2+ binding to both antigen and antibody. J Biol Chem, 1988. 263(2): p. 826-32.
Wiederschain, G., S. Raghavan, and E. Kolodny, Characterization of 6-hexadecanoylamino-4-methylumbelliferyl-beta-D-galactopyranoside as fluorogenic substrate of galactocerebrosidase for the diagnosis of Krabbe disease. Clin Chim Acta, 1992. 205(1-2): p. 87-96.
Deane, J. E. et al., Insights into Krabbe disease from structures of galactocerebrosidase, Proc Acad Sci USA, 2011. 108(37):15169-15173, supplemental materials available at pnas. 1105639108_si.pdf].

What is claimed is:

1. A composition comprising whole or truncated human wild type GALC comprising at least one of the following amino acid substitutions: T145 to 1145, T177 to R177, S200 to P200, I208 to E208, S232 to P232, K261 to W261, Y300 to N300, and C322 to S322.

2. The composition of claim 1, wherein the composition comprises the amino acid sequence of SEQ ID NO. 2.

3. The composition of claim 1, wherein the composition comprises increased GALC enzyme activity.

4. The composition of claim 3, wherein the increased GALC enzyme activity comprises at least 2-fold higher GALC enzyme activity compared to wild-type.

5. A composition comprising whole or truncated human wild type GALC of amino acid SEQ ID NO. 1 comprising one or more of the following amino acid substitutions: T177 to R177, S200 to P200, 1208 to E208, S232 to P232, K261 to W261, Y300 to N300, and C322 to S322.

6. The composition of claim 5, wherein the composition comprises amino acid substitutions of S200 to P200 and S232 to P232.

7. The composition of claim 6, wherein the composition comprises increased GALC enzyme activity.

8. The composition of claim 7, wherein the increased GALC enzyme activity comprises around 2-fold to 9-fold higher GALC enzyme activity compared to wild-type GALC.

9. The composition of claim 5, wherein the composition comprises amino acid substitution Y300 to N300.

10. The composition of claim 9, wherein the composition comprises one additional glycosylation site compared to wild-type human GALC.

11. The composition of claim 5, wherein the composition comprises one or more amino acid substitutions selected from: T177 to R177, 1208 to E208, K261 to W261, and Y300 to N300.

12. The composition of claim 11, wherein the composition comprises stabilized enzyme structure compared to wild-type human GALC.

13. The composition of claim 5, wherein the composition comprises one or more amino acid substitutions of S200 to P200, S232 to P232, and S232 to P232.

14. The composition of claim 13, wherein the composition comprises a more rigid active site loop compared to wild-type human GALC.

15. The composition of any of claims 1-14, wherein the wild-type human GALC comprises the amino acid sequence of SEQ ID NO. 1.


SEQUENCE LISTING (5 sequences)

SEQ ID NO: 1 - protein, 685 aa, Homo sapiens
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 2 - protein, 685 aa, synthetic construct
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNIILIGLPWSFPGWLGKGFDWPYVNLQLTAYYVVRWIVGAKRYHDLDIDYIGIWNERPYNANYIKELRKMLNYQGLQRVKIIASDNLWEPISASMLLDAELFKVVDVIGAHYPGTHSAWDAKLTGKKLWSSEDFSTLNSDMGAGCWGRILNQNYINGNMTSTIAWNLVASYYEQLPYGRSGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 3 - DNA, 824 bp, synthetic construct
  source 1..824: mol_type = other DNA
acgctagccaccatggctgagtggctactctcggcttcctggcaacgccgagcgaaagctatgactgctgcagctggttcggcaggaagagctgctgtgcccttgctgctgtgtgcgctgctggctcctggtggagcatacgtgctcgacgactccgacgggctgggccgggagttcgacggcatcggcgcggtcagcggcggcggggcaacctcccgacttctagtaaattacccagagccctatcgttctcagatattggattatctctttaagccgaattttggtgcctctttgcatattttaaaagtggaaataggtggtgatgggcagacaacagacggcactgagccctcccacatgcattatgcactagatgagaattatttccgaggatacgagtggtggttgatgaaagaagctaagaagaggaatcccaatattatcctcattgggttgccatggtcattccctggatggctgggaaaaggtttcgactggccttatgtcaatcttcagctgactgcctattatgtcgtgagatggattgtgggcgccaagcgttaccatgatttggacattgattatattggaatttggaatgagaggccatataatgccaattatattaaggagttaagaaaaatgctgaattatcaaggtctccagcgagtgaaaatcatagcaagtgataatctctgggagcccatctctgcatccatgctccttgatgccgaactcttcaaggtggttgatgttataggggctcattatcctggaacccattcagcatgggatgcaaagttgactgggaagaagctttg

SEQ ID NO: 4 - DNA, 443 bp, synthetic construct
  source 1..443: mol_type = other DNA
aagaagctttggtcttctgaagactttagcactttaaatagtgacatgggtgcaggctgctggggtcgcattttaaatcagaattatatcaatggcaatatgacttccacaatcgcatggaatttagtggctagttactatgaacagttgccttatgggagaagcgggttgatgacggcccaggagccatggagtgggcactacgtggtagaatctcctgtctgggtatcagctcataccactcagtttactcaacctggctggtattacctgaagacagttggccatttagagaaaggaggaagctacgtagctctgactgatggcttagggaacctcaccatcatcattgaaaccatgagtcataaacattctaagtgcatacggccatttcttccttatttcaatgtgtcacaacaatttgccacctttgttcttaaggg

SEQ ID NO: 5 - protein, 16 aa, synthetic construct
SGRGEDQVDPRLIDGK